(19) United States
(12) Patent Application Publication
(10) Pub. No.: US 2023/0294514 A1
(43) Pub. Date: Sep. 21, 2023

(54) SYSTEM AND METHOD FOR MONITORING A STATE OF A DRIVER

(71) Applicant: UNIVERSITY OF NORTH TEXAS, Dallas, TX (US)

(72) Inventors: Saraju P. MOHANTY, Dallas, TX (US); Elias KOUGIANOS, Dallas, TX (US); Laavanya RACHAKONDA, Dallas, TX (US)

(21) Appl. No.: 18/185,717

(22) Filed: Mar. 17, 2023

Related U.S. Application Data
(60) Provisional application No. 63/321,518, filed on Mar. 18, 2022.

(51) Int. Cl. B60K 28/06; G06V 20/59; G01N 33/497

(52) U.S. Cl. CPC B60K 28/063; G06V 20/597; G01N 33/4972

(57) ABSTRACT

A system for determining a blood alcohol concentration of an individual includes one or more sensors configured to measure a physiological parameter of an individual, and an analysis unit, and a method for monitoring a state of a driver related thereto. A system for determining a blood alcohol concentration of an individual can include one or more sensors, and an analysis unit configured to receive one or more outputs of the one or more sensors. The one or more sensors are configured to measure a physiological parameter of an individual, and the analysis unit is configured to determine a mental state or a physical state of the individual based on the one or more outputs of the one or more sensors.

### CROSS-REFERENCES TO RELATED APPLICATIONS

This application claims the benefit of U.S. Provisional Application No. 63/321,518, filed on Mar. 18, 2022, and entitled “BACTMobile: A SMART BLOOD ALCOHOL CONCENTRATION TRACKING MOBILE FRAMEWORK IN SMART VEHICLES USING IoMT,” which is incorporated herein by reference in its entirety for all purposes.

### STATEMENT REGARDING FEDERALLY SPONSORED RESEARCH OR DEVELOPMENT

None.

### BACKGROUND

Driving Under Influence (DUI) or Driving While Intoxicated or Impaired (DWI) is a serious offense. Though these two terms have different meanings, the offense committed by the driver is the same. The alcohol's effects on the human body starts the moment someone consumes their first sip. Prolonged alcohol consumption effects involve not only behavioral changes, blackouts, slurred speech, fatigue, malnutrition, infertility, addiction, and numbness but also involve damage to vital organs such as the liver, the heart, and can cause diabetes and lung infections. The immediate effect of alcohol on the human body includes slurred speech, drowsiness, vomiting, headaches, loss of consciousness, memory lapse and distortion of senses and perception.

The alcohol percentage in a person's bloodstream is known as Blood Alcohol Concentration (BAC). As BAC increases, the level of intoxication of a person increases. BAC of 0.08% is determined as the legal limit a person can drive in most states of the US. BAC of 0.10% indicates that the blood supply in an individual contains one part of alcohol for every 1000 parts of blood. The number of drinks, amount of time in which they are consumed, body weight, age, and sex are among the various factors that are considered in calculating BAC.

Road accidents are increasing at a significant rate every year. There are nearly 40,000 fatal car accidents recorded in the United States per year. There are more than 90 Americans dying in car accidents per day. Out of these 40,000 accidents, 40% are due to drunk driving, 30% are due to speeding and 33% are due to irresponsible driving.

The growth in science and technology is adding intelligence to all components of life, making it “Smart-life”. The key point of Smart-Life is the ability to communicate with all the other components, which in turn is known as the Internet of Things (IoT). The Internet of Things (IoT) is defined as a network of interrelated, connected devices, mechanical, digital machines, or objects that are provided with a unique IP address for easy exchange of information without requiring human-to-human or human-to-computer interaction.

A major component of Smart-Life will be Smart-Cities. Smart-Cities are comprised of Smart-Healthcare, Smart-Buildings, and Smart-Transportation, otherwise known as Smart-Cars. There has been immense growth in the automobile technology where the invention of driver-less cars is also possible. Generally, there is a desire to provide a system to minimize risks associated with drivers and driving.

### SUMMARY

The systems and methods described herein overcome many shortcomings of the problems associated with unreliable drivers and driving. The problems overcome include: failing to automatically monitor blood alcohol concentration levels of the driver; not having a mechanism to detect the behavior of the driver throughout the driving period; requiring an external officer or a person to detect the BAC levels of the driver; requiring an external officer or a person to detect the mental state of the driver; failing to consider other physiological parameters which could help in analyzing the mental state of the driver; failing to consider false negative cases by not having continuous monitoring; failing to provide a feasible, simple communication method with family or emergency services when in need; failing to consider the worst-case scenario where the driver loses consciousness to book a cab or ask for help; failing to provide a cost-effective solution which enables the user to make the most of the device; not having restricted location mechanisms; failing to provide constant care by continuously monitoring the vitals throughout the driving period; and failing to monitor the psychological features of the driver to analyze the mental state of the driver over a certain period. What is more, further problems include: not having additional data to validate the state of the driver; not having a mechanism to allow the user to know the detected BAC levels; failing to provide medical support in need irrespective of the location of the user; failing to have a relationship among the physiological parameters in humans to the blood alcohol concentration levels; failing to have a relationship among the psychological parameters in humans to the blood alcohol concentration levels; failing to have an ability to analyze the facial parameters of the person to correlate with blood alcohol concentration levels; failing to incorporate an alcohol sensor inside the vehicle system to monitor the blood alcohol levels; failing to monitor the physiological and facial parameters of the driver who is driving long distances; failing to have continuous monitoring systems for physically impaired drivers; failing to have a single, compact solution which has a capability to connect to any network or any device in the network; and failing to have a solution with the safety and comfort of the user in mind.

The systems and methods of embodiments disclosed herein can provide a method for proposing a system which performs automated multimodal, multidata, continuous monitoring of the physiological, facial, and psychological parameters throughout the driving period of the drivers to accurately analyze the mental state and the driving ability; a method to respond using psychological parameter analysis to eradicate the false positive cases; a method with a device to enable two-way communication when needed; a method to provide a device that has action-response systems incorporating audio and touch for intoxicated drivers; a method of suggesting control mechanisms to reduce the chances of occurrences of accidents; a method to warn the driver when there is an abnormality detected during longer period driving; a method to predict the occurrence of accidents and alerting the driver accordingly; a method for automatic vital monitoring for drivers throughout the driving period to analyze the behavioral and physiological changes; an approach for proposing a battery-operated wearable device that recharges with the engine's working; a method for proposing multidevice interactions using the Internet of Things (IoT) for providing a system to analyze both physiological and visual data; a method to provide individual cyber physical systems on-site in each vehicle for immediate attention in case of a situation where there is a disconnection; and a method to understand the difference between fatigue and intoxicated states of the driver.

Furthermore, the systems and methods of embodiments disclosed herein can provide a method to propose a system for monitoring the vital signals and can analyze the stress levels and other emotions associated with stress of older adult drivers that may lead to accidents; a method that has the automatic enabling of a panic button for help instead of waiting for the user to activate allowing panic button activation in case of an unconscious state of the driver in case of, e.g., an accident; a method to connect through IoT to the database and nearby help units depending upon the location of the vehicle in case of an emergency; a method to provide a physical system that not only monitors the multimodal data but also records the behavior of the driver, contains a panic button, and has a mechanism to let the user know when in danger using an infotainment; a method with a physical embedded system that can analyze the psychological behaviors of the driver using the infotainment; a method for a system that stops the occurrences of the accidents by having an automatic ignition lock for the vehicle; a method has a system that first analyzes the physiological parameter changes, facial feature changes and analyzes the blood alcohol concentration of the driver; a method has a system that allows the driver to give feedback for the decision made by the system, thereby having a chance to analyze the psychology of the driver; a method has a system that makes an informed decision whether to let the driver drive the vehicle or not by considering various data; a method that provides an option for the driver to contact for help or family or other means of transport through IoT using infotainment as the interface; a method that not only does continuous location detection and tracking but also shares that information to the nearest emergency in case of an accident; and a method has a system that allows automated continuous monitoring and recording of the circumstances to analyze the situation of an accident more accurately and efficiently. Additionally, the systems and methods of embodiments disclosed herein can provide a method to consider more physiological parameters other than just breath of the driver to increasing the efficiency; a method considers ethanol sensor readings to eliminate false positive cases; a method collectively sends all the data to help when in need; a method automatically suggests drivers with control mechanisms after the ignition locks; a method notifies the nearest emergency respondent when there is a sudden change in physiological and vision data to prevent accidents; a method continuously monitors the vital signals of the user to predict major health hazards like heart attacks, diabetic unconsciousness, or low blood oxygen levels; a method has a voice enabled system to alert the driver in accident-prone areas; a method to check if the driver along with the vehicle is safe or moving by continuously monitoring the location of the user by having automated and continuous location tracking; a method continuously has the visuals of the driver when driving to analyze the alertness; a method has an approach where the driver can communicate with the emergency responders through the infotainment center that is in the network, thereby not obviating the use of a phone when in danger; a method has a system not only for older adult drivers, but for anyone with disabilities; and an Internet-of-Medical-Things (IoMT) based Healthcare Cyber-Physical System (H-CPS) framework with three different devices and approaches can analyze the mental health of the driver to prevent the occurrences of accidents.

Additionally, the systems and methods can provide a non-invasive, automated real time monitoring system activated by human touch or when the car ignition starts. Moreover, the method does not require human-to-human interaction. Furthermore, the system can predict BAC and check if the driver is in inebriated or not throughout the driving period so as to reduce accidents. Also, the system can detect the exact BAC with five intervals in order to educate the driver of his driving capability. What is more, the analyzed physiological signal data may be saved to the cloud, e.g., Internet, for future reference and the notification is displayed on an infotainment. The system can provide both in-network and out-of-network privacy to assure secure data transfer throughout the driving period. Usually, a system using a novel algorithm focused in obtaining high throughput, reliability and low power usage to establish a secure connection among entities.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 is a schematic of an embodiment of a system.

FIG. 2 is a schematic of an embodiment of a communication system of the system.

FIG. 3 is a schematic of an embodiment of various components of the system.

FIG. 4 is a schematic of an embodiment of an analysis unit of the system.

FIG. 5 is a schematic of an embodiment of another analysis unit of a system.

FIG. 6 is a flow diagram of an embodiment of a method.

FIG. 7 is a flow diagram of an embodiment of a step for collecting images/videos of the method.

FIG. 8 is a flow diagram of an embodiment for a step of raw input multimodal data of the method.

FIG. 9 is a flow diagram of an embodiment for a step of processed input physiological and vital data.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

A system and method uses an edge-level solution through the of Things (IoT). The system has the ability to monitor the vitals, physiological and facial features of the driver to determine the Blood Alcohol Concentration (BAC) levels. The system can not only track the BAC levels, but it can also analyze the mental health of the driver by accepting and analyzing the psychological data using the infotainment center of the vehicle. The psychological data can be used to eliminate the possibilities of false positive cases. The system can lock the ignition of the car based on the results of multimodal data analysis.

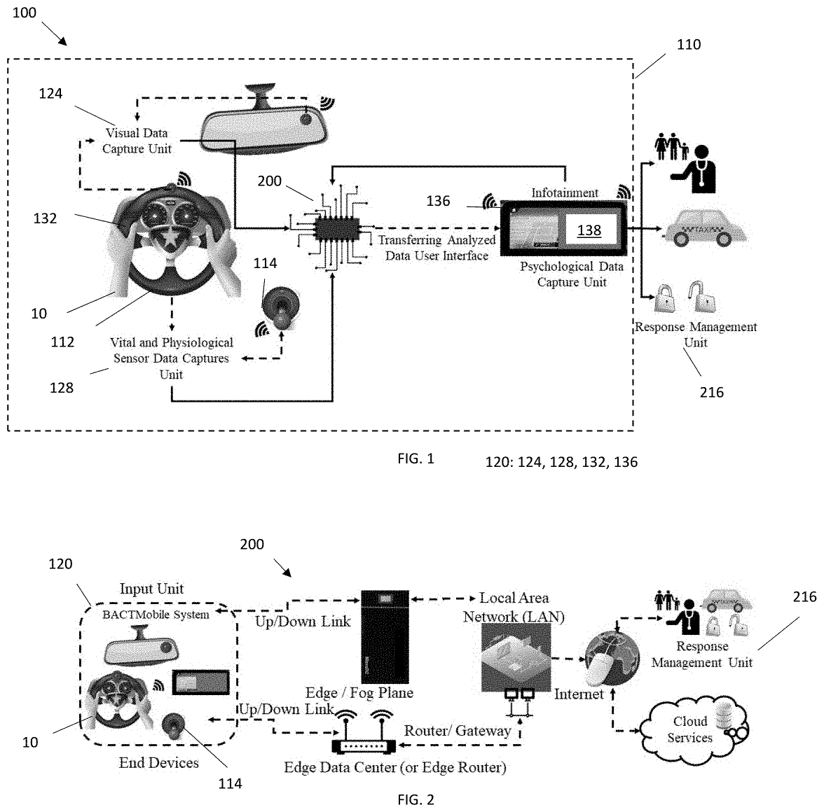

In some embodiments, the system is a component in a vehicle as depicted in FIG. 1. Referring to FIGS. 1-3, a system 100 for determining BAC of a driver, such as an individual 10, includes a vehicle 110 having a steering wheel 112 and an ignition 114, one or more sensors 120, an analysis unit 180, and a communication system 200. A response management unit 216 may be responsive to the system 100, as described hereinafter. The one or more sensors 120 can be configured to measure a physiological parameter of an individual, and can include an image capture sensor 124, a vital data sensor 128, a medical sensor 132, a psychological monitoring sensor 136, or any combination thereof. The image capture sensor 124 can be configured to output at least one of an image or a video. The medical sensor 132 can be a physiological sensor. The vital data sensor 128 can be configured to output at least one of a respiration rate, an electroencephalogram output, a temperature reading, a blood pressure reading, a heart rate, a skin conductance reading, a blood oxygen level, a blood sugar level, or a combination thereof. The psychological monitoring sensor 136 can be incorporated into an infotainment counsel 138 of the vehicle 110 and configured to output at least one of a movement output, an accelerometer output, a gyroscopic output, a pressure output, a body position output, a light detection and ranging (LIDAR) output, a location reading, a blood alcohol reading, or a movement of a chest or abdomen of the individual 10. The one or more sensors 120 can include an input unit for receiving stimulus. The one or more sensors 120 can provide an output to the analysis unit 180 configured to receive one or more outputs of the one or more sensors 120.

The analysis unit 180 may be configured to determine a mental state or a physical state of the individual 10 based on the one or more outputs of the one or more sensors 120. The analysis unit 180 can include a microcontroller and a machine learning model (MLM), including one or more MLMs, such as a neural network (e.g., a tiny deep neural network model), configured to accept the one or more outputs of the one or more sensors 120, and predict a physical state of the individual 10.

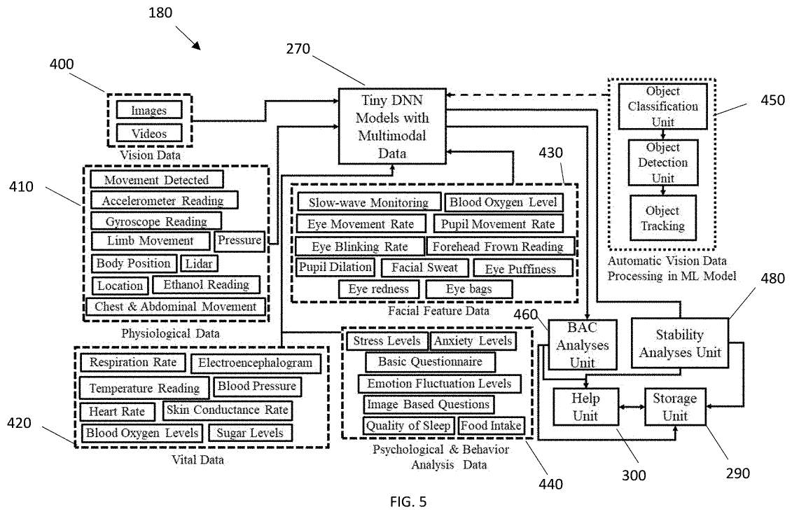

In some aspects, a MLM architecture can be represented in the form of a neural network having layers and neurons that have been used for the process of multimodal data analysis. An embodiment of a MLM is as shown as in FIG. 4. As depicted in FIG. 4, the analysis unit 180, such as a neural network, can include multiple layers, such as an input layer 320, one or more hidden layers 340, and an output layer 360. A fully connected neural network (FCNN) model with a linear stack of one input layer, three hidden layers and one output layer with 10 neurons each can be used. The data training methodology is explained through the following algorithm. The training steps for a tiny deep neural network (DNN) can be as follows: 1) set the epoch value. Iterate each stress epoch. This epoch defines the number of times the dataset to loop; 2) inside every repetition, iterate every example from training dataset by correlating its input features, e.g., the physiological, vital, psychological parameters and the output labels, e.g., the BAC levels; 3) using these features and training dataset, make inferences; 4) compare the actual BAC level outputs with the BAC predictions from the previous step; 5) calculate loss at every epoch; 6) calculate the training data loss and accuracy in order to determine the overall efficiency; 7) update the variables to predict BAC levels with the help of optimized algorithm using the Gradient Descent algorithm; and 8) repeat the above steps for all the BAC epoch count.

The physical state can include a BAC of the individual. In some embodiments, the analysis unit 180 can be configured to lock an ignition of a vehicle when the blood alcohol content of the individual 10 is above a threshold. The analysis unit 180 can include a vital signal data monitoring unit, a response management unit 216, and a blood alcohol concentration detection unit. The response management unit 216 in the system 100 has pre-stored contact numbers of the family or friends, has an ability to contact the nearest emergency service, has an ability to book a cab or call for a taxi automatically. This response system also has the access to the video recordings of the driver and can transfer it to the nearby emergency when in need in case of driver unconsciousness.

The communication system 200 can be in signal communication with the analysis unit 180. The communication system 200 may be configured to send an indication of the mental state or the physical state of the individual 10 to a remote device. The communication system 200 can be configured for two-way responses, and can include equipment and systems such as up and down links, fog computing using edge devices and peripherals in a plane traffic, edge data center or router, at least one of a router, a gateway, or a combination thereof, one or more local area networks, one or more cloud services, and the Internet. After analysis, user interface data can be transferred over the various communication links.

In some embodiments, the system for checking BAC and minimizing human intervention is to minimize the requirement for humans to check if the driver is in a stable mind to drive or not. As an automated device, the system automatically monitors the physiological signal data of the driver every time the driver touches the steering mechanism. By analyzing the physiological data, the decision whether the driver is capable of driving or not is made and actions are taken accordingly.

The system and method for determining BAC provides a non-invasive, automated real time monitoring system activated by human touch, an advanced method not requiring human-to-human interaction, monitoring physiological parameters to determine the state of a person to decide if the person can drive by the measured physiological parameters, a prediction of BAC to check if the driver is inebriated or not throughout the driving period to eliminate any scope of accidents, a detection of the exact BAC with five intervals in order to educate the driver of his driving capability, and an analysis of physiological signal data saved to the cloud for future reference and display of the notification on the infotainment.

The system and method can predict the BAC before the driver starts driving rather than in the process of driving. Any regular system can be converted to the system and method by adding an add-on device as disclosed herein. This system can be optimized as the physiological signal check is completed only during the driving period. Once the physiological signal is obtained, it can be sent to a cloud database for storing purposes. After the analysis, the decision is displayed on the car's infotainment system.

The impact of growth in science and technology has led to car companies attempting to incorporate an alcohol level detection system in smart cars that automatically determines the BAC and whether the person is able to drive. However, these companies have used the touch technology but this is limited to a certain point with no real time continuous monitoring providing a chance of misuse of the technology. On the other hand, there are wearables that are available in the market in order to test the alcohol consumption but that depends solely on the driver. There are smart phone application-based works presented in the literature that are insufficient to stop accidents.

A photoplethysmogram (PPG) signal based approach is one alternative for the blood alcohol level detection. The PPG signal data from four different individuals can be obtained and analyzed to detect the blood alcohol concentration. The system and method of some embodiments can detect the blood alcohol concentration greater or less than the 0.08%, the limit for driving in most of the states. With this, the driver's ability to drive is stated but the analyzation of driver's consciousness stability cannot be predicted. The system and method can be capable of not just detecting if BAC is less than or greater than 0.08%, but also detect the exact level of BAC as that can be useful in order to analyze the mental ability of the driver.

Detecting the blood alcohol concentration of a person is determined by using biometric scan as the key. This biometric scan can be used in the system and method as the original pupil diameter and redness in the eye can be used as baseline information for the comparison. The decision of blood alcohol concentration is not solely dependent on biometric scan, but also dependent on blood pressure data for more accurate results. The dependency on just the physiological signals is highlighted in the methods and systems, which helps in developing more robust, simplified and less costly solutions to the same problem.

Whenever the driver touches the steering wheel, the physiological signal data is taken, pre-processed and is compared with the baseline information. The data is then analyzed in the microcontroller and the decision to allow the driver to drive or not is determined and is displayed on the car's infotainment center. The IoT cloud is used as storage for the collected data and the decisions made by the system. The microcontroller acts as a “thing” as it is the key part for any car to turn it into a smart-car.

When the driver starts the engine, the touch of his hand on the steering wheel activates the sensors that are incorporated in it. The physiological data is gathered at this state. The gathered data is analyzed in the microcontroller unit which can either be placed on the steering or anywhere in the car. This way, the microcontroller acts like a part of the network. After the data is analyzed, the decision if the driver is sober or not is made and is sent to the infotainment. If the driver is sober, the driver is allowed to drive but if the driver is not sober, the engine is locked to prevent accidents. The process of gathering and analyzing the physiological data is done throughout the driving period in order to prevent any misuse of technology. The gathered, analyzed data can be sent to a database for storage.

The parameters of the physiological signals of the systems and methods along with their relationship with alcohol consumption include the following parameters: temperature, respiration rate, heart rate, and blood pressure. The immediate effects of alcohol that have short-term impact on the human body with blood alcohol consumption greater than or equal to 0.08% include increase in blood pressure, blood oxidation content, and heart rate. Increase in alcohol consumption may result in very irregular, short and slow bursts in breathing that effect the rate of respiration of the person. The increase in the content of blood alcohol also have impacts on the temperature of the body. The temperature rises because of the distribution of heat in the body and it makes the body feel warm.

Photoplethysmogram (PPG) signal data can be observed for respiration and blood pressure. The observed results also had a relationship with the intake of alcohol typically till the level reached 0.08% of BAC. The established relationship with the BAC content to the PPG signal data is represented by the following expression:

m*nl>m*nh (1)

m*nl<m*nh, (2)

In the above expressions, Equation (1) represents low alcohol consumption level, e.g. BAC<0.08%, Equation (2) represents high alcohol consumption level, e.g., BAC>0.08%, m is the PPG signal data, nl is the low alcohol consumption level.

The temperature and heart-rate can be obtained from literature published data. The normal body temperature and resting heart-rate of the human body are 97-99° F. and 60-100 beats per minute. If there is a difference in the measured values to the baseline values, the person is assumed to be consuming alcohol. The sensor signal calibration data are represented in Table I while the classification of BAC levels is represented in Table II.

For the system implementation of the system and method, the dataset based on Table II is taken and is executed in a programming and numeric computing platform, such as MATLAB® computing platform available from MathWorks, Inc. of Natick, Massachusetts (hereinafter “MATLAB®”). As an example, a reading of 0.02 not only represents if the person has exceeded the state rule of BAC but also determines the exact percentage of BAC so as to observe the driver's behavior. The system and method can be implemented with open-source firmware such as NodeMCU or a self-contained system and organization control (SOC) with integrated transmission control protocol/Internet protocol (TCP/IP) stack such as ESP8266 along with the physiological sensors. The analyzed outputs can be directly sent to the cloud for storage. The temperature, respiration, blood pressure, and heart rate sensors can be used to obtain the sensor information, and the system and method analyzes the data with an average delay of 10 milliseconds. The decision is represented in the infotainment center using the LED display. The system can produce an accuracy of approximately 93% when trained with set of 256 rules which have various combinations of the four physiological signal data.

TABLE I
SENSOR SIGNAL CALIBRATION

| Sensor/ | Feature | | BAC |
|---|---|---|---|
| Signal Type | Considered | Baseline Condition | Conditions |
| Heart Rate | Heart Rate | 60-90 beats/min | >90 beats/min |
| Sensor | | | |
| Temperature | Temperature | 97-99° F. | >99° F. |
| Sensor | | | |
| PPG Signal | Respiration Rate, | 12-20 breaths/min, | m*nl>l<m*nh |
| Data | Blood Pressure | 110/70 to 120/80 | |

TABLE II
BAC LEVEL REPRESENTATION

| | Respiration | | | | |
|---|---|---|---|---|---|
| PPG Signal | Rate | Blood | HeartRate | | BAC |
| Data | (breaths/min) | Pressure | (beats/min) | Temperature | (%) |
| nl = 0.994, | 12-20 | 110/70- | 60-90 | 97-99 | -Sober |
| nh = 0.966 | | 120/80 | | | |
| nl = 0.248, | 10-12 | 120/80- | 90-100 | 99-101 | 0.02 |
| nh = 0.2415 | | 125/85 | | | |
| nl = 0.497, | 9-10 | 125/85- | 100-105 | 101-102 | 0.04 |
| nh = 0.483 | | 130/85 | | | |
| nl = 0.745, | 5-9 | 135/85- | >105 | >102 | 0.06 |
| nh = 0.724 | | 140/90 | | | |
| nl = 0.994, | <5 | >140/90 | >105 | >102 | 0.08 |
| nh = 0.966 | | | | | |

The systems and methods detect blood alcohol concentration level of a person through a smart device before the process of driving starts. This device can turn a regular device to a smart device by connecting to the Internet to store the data. As soon the driver touches the steering wheel, the physiological sensors get activated and collect the initial data of the driver. This obtained data is checked with the regular baseline information and a decision of the driver's sobriety is made. If the driver is sober, he is allowed to drive but if he is not, the car will automatically lock the engine along with the blood alcohol concentration information being displayed on the infotainment. The exact blood alcohol concentration present in the human body is also determined. The information is processed in a microcontroller with an approximate accuracy of 93% and the data is sent to an Internet of things analytic tool which can be accessed by the user later.

Additionally, the impact of physiological signal data can observe the changes in physiological parameters during various chores of a person. Also, the type, amount and time of food consumption, the average sleep score, the physiological parameter changes during these actions to can make a significant improvement not only in the detection of blood alcohol consumption but also to analyze person's behaviors an aspect.

For the cloud connectivity, ThingSpeak, an IoT Analytics tool, can be used where the data collected in the cloud can be analyzed using MATLAB®. The combined intoxicated and sober signal data along with the time of occurrence is represented in ThingSpeak in a 2-D graphical format.

Alcohol consumption detection can depend on only one sensor signal data with the drawback that the results have noticeably less accuracy. The system and method uses more than one physiological signal data to not only identify the individual level of alcohol blood concentration, but also state if the user is sober.

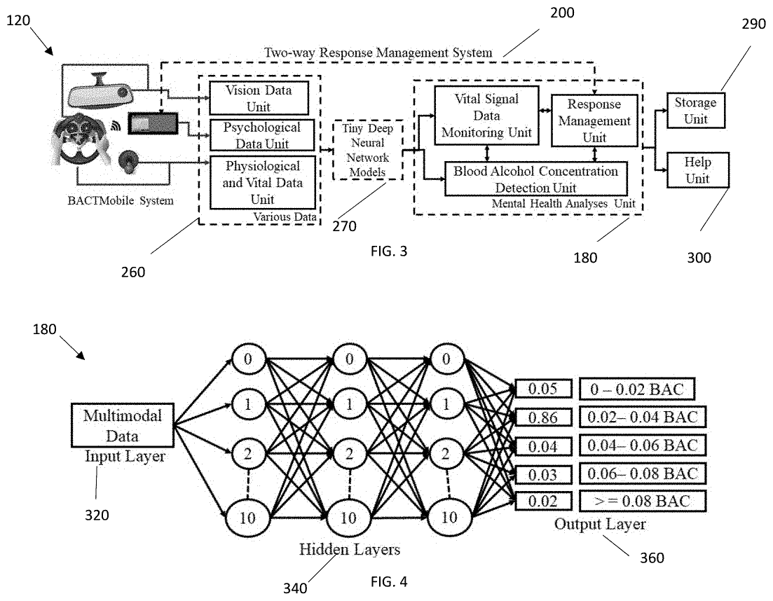

Turning back to the system 100, as depicted in FIG. 3, the system 100 can further include a data unit 260, which can include a vision data unit, psychological data unit, and physiological and vital data unit, a storage unit 290, and a help unit 300.

In some embodiments, as depicted in FIG. 5, an analysis unit 180, which may include a mental health analyses unit, can include the tiny DNN models with multimodal data 270, which may also or alternatively include the tiny DNN, as described above, a vision data unit 400, a physiological data unit 410, a vital data unit 420, a facial feature data unit 430, a psychological and behavior analysis data unit 440, a MLM 450, a BAC analysis unit 460, the storage unit 290, the help unit 300, and a stability analysis unit 480. The various modal data is collected and is sent to the BAC unit 460 and the stability analyses unit 480 for accurate BAC detection and accident prevention. The automated processing is performed in the MLM. The tiny DNN models with multimodal data 270, the storage unit 290, and the help unit 300 are discussed above.

The vision data unit 400 can include image and video data. The physiological data unit 410 can include movement detected data, accelerometer reading data, gyroscope reading data, limb movement data, pressure data, body position data, LIDAR data, location data, ethanol reading data, and chest and abdominal movement data. The vital data unit 420 can include respiration rate data, electroencephalogram data, temperature reading data, blood pressure data, heart rate data, skin conductance rate data, blood oxygen levels data, and sugar levels data.

The facial feature data unit 430 can include slow-wave monitoring data, blood oxygen level data, eye movement rate data, pupil movement rate data, eye blinking rate data, forehead frown reading data, pupil dilation data, facial sweat data, eye puffiness data, eye redness data, and eyebags data. The psychological and behavior analysis data unit 440 can include stress levels data, anxiety levels data, basic questionnaire data, emotion fluctuation levels data, image based questions data, quality of sleep data, and food intake data. The MLM 450 for automatic vision data processing can include an object classification unit, an object detection unit, and an object tracking unit.

In some embodiments, the data from the input unit can be taken and analyzed by a microcontroller. This data can be sent to the tiny DNN model where it is analyzed, and the appropriate BAC levels are predicted. At this phase, if the driver 10 feels the need to negate the response given by the system 100, the driver 10 has an option to answer a few basic questions which help to analyze the psychological and mental state of the person. If the driver 10 is not in a good mental health state to drive, the ignition 114 will be locked. All the information along with the parameters analyzed are transferred to the cloud for storage purposes. Additionally, the decisions and the condition of the driver 10 are sent to the family or for help through response management unit 216 as shown in FIGS. 1-2.

The physiological, vital, psychological along with the camera input data can be taken from the driver 10 and analyzed at the edge level processing unit. This processed data is sent to the family and doctor for help, depending on the emergency.

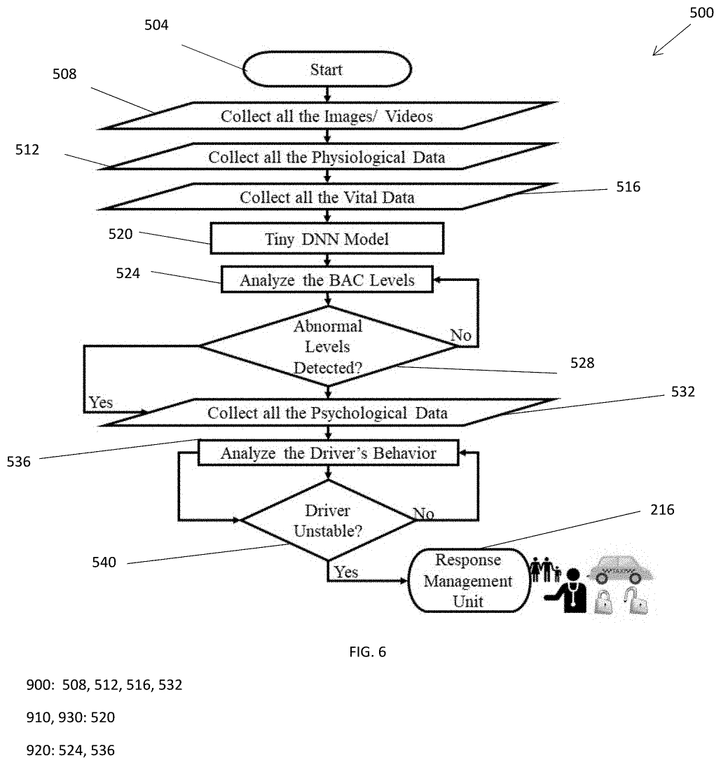

Referring to FIGS. 6-9, a method of monitoring 500 overall working flow as a complete framework is specifically depicted in FIG. 6. The method of monitoring 500 can begin at “start” at the step 504. Next, collect all the images/videos at a step 508, collect all the physiological data at a step 512, and collect all the vital data at a step 516. After collecting, the data can be outputted to a tiny DNN model at a step 520. Next, analyze the BAC levels at a step 524. That being done, a query of whether abnormal levels detected is conducted at a step 528. If no, then the step 524 repeated. If yes, then collect all the psychological data is conducted at a step 532. Next, analyze the driver's behavior is conducted at a step 536. Another query is made whether the driver is unstable at a step 540. If no, step 536 is repeated, and if yes, the response management unit 216 is notified at a step 216. Moreover, the step 900 of monitoring at least one of a physiological parameter, a facial feature, or a psychological parameter of a driver using one or more sensors disposed within a vehicle can include steps 508, 512, 516, and 532. Furthermore, the steps receiving, by an analysis unit 180, at least one output of the one or more sensors 910 and inputting the at least one output into a MLM 930 can be done at the step 520 for using the tiny DNN model. What is more, the steps of determining, by the analysis unit, a mental state or a physical state of the driver based on the at least one output 920 can include steps 524 and 536.

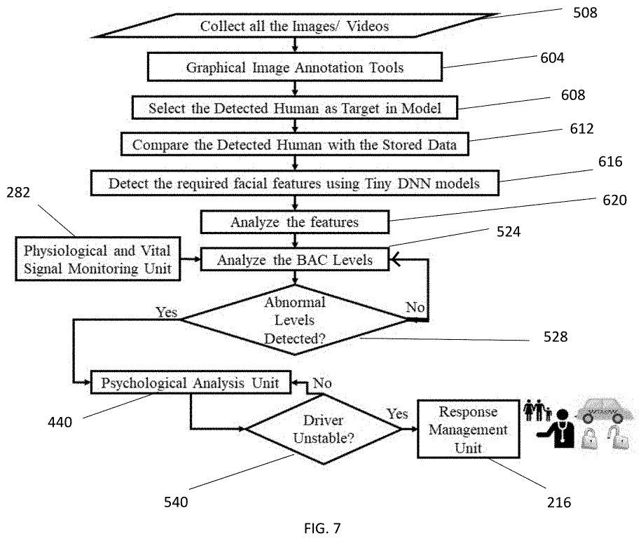

As the driver starts the car, the cameras attached can capture all the image and video data along with physiological and vital signal data. The complete data will be sent to the tiny DNN model where the BAC levels can be detected. Based on how the driver responds, the psychological data is taken and is analyzed to monitor the driver's ability to drive the vehicle. The process of taking the images and converting them to analyze the BAC is listed in Algorithm 2 below. The same process is represented through FIG. 7. The algorithm 2 can include: 1) images used for testing and training the model are collected; 2) the formats of the images are converted from JPEG to XML after creating bounding boxes by using a graphical image annotation tool; 3) multiple bounding boxes in various images for the same feature, which are called priors are also created in the same annotation tool; 4) using box-coder, the dimensions of priors are made equal; 5) by considering the concept of IOU (Intersection Over Union), the matched and unmatched thresholds for matching the ground truth boxes to priors are set. This is highly desirable as the model may not be ready for training if the match has not been made; 6) images in XML, format are made equal in size either by using reshape or resize functions; 7) using convolution and rectified linear functions, the feature maps are assigned to every image sent to the model; 8) based on these features, the images are either sent to regression or classification where the objects are detected through boxes in the images; and 9) repeat the above steps for all the images.

Referring to FIG. 7, a subroutine to collect all images and videos begins at a step 508. Next, graphical image annotation tools can be activated at a step 604. That being done, select the detected human as target in model can be done at a step 608. Afterwards, compare the detected human with the stored data is completed at a step 612. Next, detect the required facial features using tiny DNN models at a step 616 and analyze the features at a step 620 is completed. The step 524 analyze the BAC levels can receive the output from the step 620 and physiological and vital signal monitoring unit 282 to, in turn, output to a query of abnormal levels detected at a step 528. If no, return to step 524, otherwise if yes, proceed to psychological analysis unit at a step 440. Another query can be made of the driver unstable at a step 540. If no, return to the step 440, otherwise if yes, the response management unit 216 can be notified at a step 216.

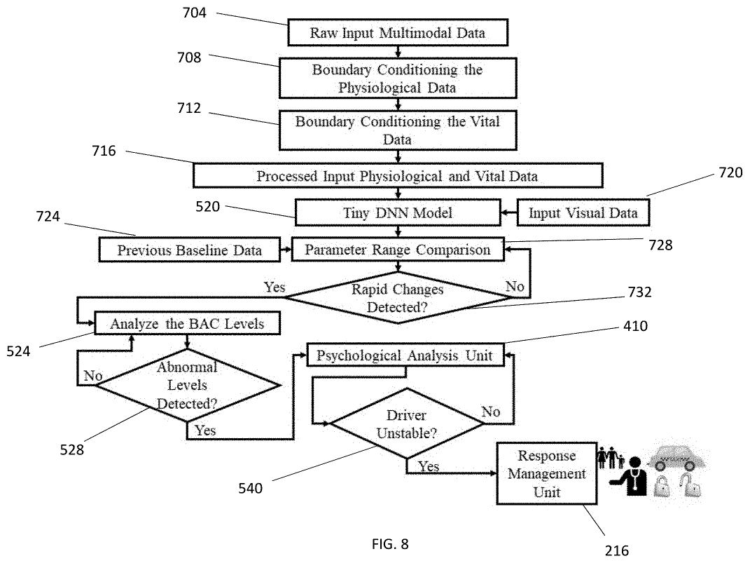

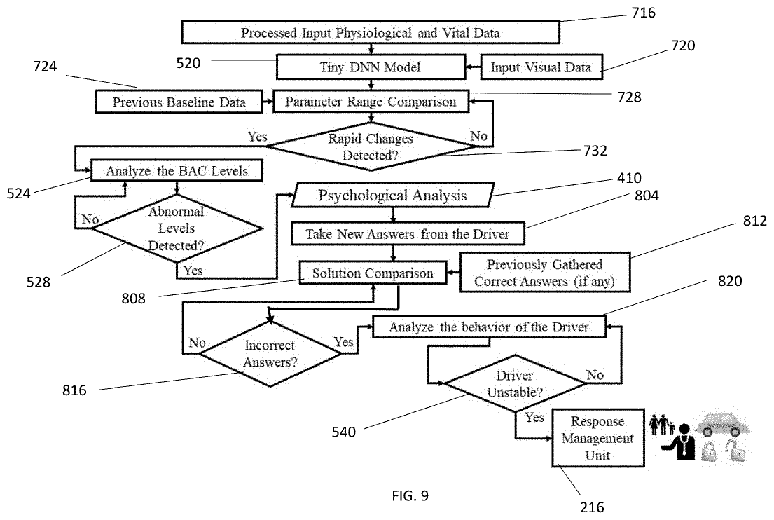

Referring to FIG. 8, a subroutine can raw input multimodal data at a step 704. Next, boundary conditioning the physiological data may occur at a step 708. That being done, boundary conditioning the vital data at a step 712 and processed input physiological and vital data can be done at a step 716. A tiny DNN model at a step 520 can receive the output from the step 716 and input visual data from a step 720. A parameter range comparison at a step 728 can receive the output from the step 520 and the output from previous baseline data at a step 724. After the parameter range comparison at the step 728, a query of rapid changes detected can be done at a step 732. If no, then step 728 is repeated, otherwise if yes, analyze the BAC levels at a step 524. That being done, another query can be made whether abnormal levels are detected. If no, step 524 is repeated. If yes, then a psychological analysis unit can be completed at a step 410. Afterwards, another query can be made, namely, driver unstable at a step 540. If no, then step 410 is repeated, if yes, then the response management unit 216 can be contacted at a step 216.

The automatic flow of the psychological data to analyze the stability of the driver is represented in FIG. 9. All the multimodal data—physiological, vital, image and video—are considered to analyze the BAC levels of the person. Referring to FIG. 9, another subroutine is depicted. Initially, processed input physiological and vital data can occur at a step 716. Next, a tiny DNN model can be activated at a step 520 and receive output from input visual data at a step 720 and the step 716. The output from step 520 can be received for a parameter range comparison at a step 728 that also receives a previous baseline data output from a step 724. The output from the step 728 can be queried for rapid changes detected at a step 732. If no, then the step 728 is repeat, if yes, then analyze the BAC levels at a step 524. The output from the step 524 can be queried for abnormal levels detected at a step 528. If no, step 524 is repeated, if yes, then psychological analysis is conducted at a step 410. Next, take new answers from the driver at a step 804 and then solution comparison at a step 808 that receives an output from previously gathered correct answers (if any) at a step 812. The output from the step 808 can be queried, namely, incorrect answers at a step 816. If no, then step 808 can be repeated, if yes, then analyze the behavior of the driver at a step 820. The output of the step 820 can be queried if the driver unstable at a step 540. If no, then the step 820 is repeated, if yes, then the response management unit 216 can be contacted at the step 216.

In case of a false positive, the driver is asked with a certain questionnaire which has a combination of known and unknown (logical) thinking questions. This questionnaire can even consider image data and ask the user to identify the family or friend along with other behavior analysis questions. Doing this, will allow the system 100 to reanalyze the mental state of the driver and the system can make an informed decision to either unlock the ignition or to allow the user to use the response management unit 216.

Additional advantages may be apparent to one of skill in the art viewing this disclosure.

Having described various systems and methods herein, certain embodiments can include, but are not limited to:

In a first aspect, a system for determining a blood alcohol concentration of an individual, the system comprises: one or more sensors, wherein the one or more sensors are configured to measure a physiological parameter of the individual; and an analysis unit configured to receive one or more outputs of the one or more sensors, wherein the analysis unit is configured to determine a mental state or a physical state of the individual based on the one or more outputs of the one or more sensors.

A second aspect can include the system of the first aspect, further comprising a vehicle, wherein the one or more sensors and the analysis unit are part of the vehicle.

A third aspect can include the system of the first or second aspect, wherein the one or more sensors comprise an image capture sensor, a vital data sensor, a medical sensor, or a psychological monitoring sensor.

A fourth aspect can include the system of any of the preceding aspects, wherein the one or more sensors comprise an image capture sensor, and wherein the image capture sensor is configured to output at least one of an image or a video.

A fifth aspect can include the system of any of the preceding aspects, wherein the one or more sensors comprise a physiological sensor, and wherein the physiological sensor is configured to output at least one of a movement output, an accelerometer output, a gyroscopic output, a pressure output, a body position output, a LIDAR output, a location reading, a blood alcohol reading, or a movement of a chest or abdomen of the individual.

A sixth aspect can include the system of any of the preceding aspects, wherein the one or more sensors comprise a vital data sensor, and wherein the vital data sensor is configured to output at least one of a respiration rate, an electroencephalogram output, a temperature reading, a blood pressure reading, a heart rate, a skin conductance reading, a blood oxygen level, or a blood sugar level.

A seventh aspect can include the system of any of the preceding aspects, further comprising: a communication system in signal communication with the analysis unit, wherein the communication system is configured to send an indication of the mental state or the physical state of the individual to a remote device.

An eighth aspect can include the system of any of the preceding aspects, wherein the analysis unit comprises a neural network configured to accept the one or more outputs of the one or more sensors, and predict the physical state of the individual.

A ninth aspect can include the system of any of the preceding aspects, wherein the physical state comprises a blood alcohol content of the individual.

A tenth aspect can include the system of any of the preceding aspects, wherein the analysis unit is further configured to lock an ignition of a vehicle when the blood alcohol content of the individual is above a threshold.

In an eleventh aspect, a method of monitoring a state of a driver, the method comprises: monitoring at least one of a physiological parameter, a facial feature, or a psychological parameter of the driver using one or more sensors disposed within a vehicle; receiving, by an analysis unit, at least one output of the one or more sensors; and determining, by the analysis unit, a mental state or a physical state of the driver based on the at least one output.

A twelfth aspect can include the system of the eleventh aspect, wherein determining the mental state or the physical state comprises: inputting the at least one output into a machine learning model; determining a blood alcohol concentration (BAC) level of the driver as an output of the machine learning model; and outputting the BAC level of the driver.

A thirteenth aspect can include the system of the eleventh or twelfth aspect, further comprising: determining that the BAC level of the driver is above a threshold; monitoring a behavior of the driver; determining that the behavior of the driver indicates that the driver is unstable; and sending an alert to a response management unit in response to determining that the behavior of the driver is unstable.

A fourteenth aspect can include the system of any of the eleventh aspect to the thirteenth aspect, wherein the one or more sensors comprise an image capture system, and wherein determining the BAC level of the driver comprises: capturing one or more images of the driver; detecting one or more facial features of the driver within the images; determine an indication of the one or more facial features; and determining the BAC level of the driver using the one or more facial features in an analysis model.

A fifteenth aspect can include the system of any of the eleventh aspect to the fourteenth aspect, further comprising: using one or more physiological sensor outputs or one or more vital sensor outputs with the one or more facial features in the analysis model.

A sixteenth aspect can include the system of any of the eleventh aspect to the fifteenth aspect, further comprising: determining that the BAC level of the driver is above a threshold; presenting one or more questions to the driver on a display interface; receive one or more answers from the driver in response to the one or more questions; inputting the one or more answers to a psychological analysis model; and determining a psychological state of the driver based on an output of the psychological analysis model.

A seventeenth aspect can include the system of any of the eleventh aspect to the sixteenth aspect, wherein the one or more sensors comprise an image capture sensor, a vital data sensor, a medical sensor, or a psychological monitoring sensor.

An eighteenth aspect can include the system of any of the eleventh aspect to the seventeenth aspect, wherein the one or more sensors comprise an image capture sensor, and wherein the image capture sensor is configured to output at least one of an image or a video.

A nineteenth aspect can include the system of any of the eleventh aspect to the eighteenth aspect, wherein the one or more sensors comprise a physiological sensor disposed within a steering wheel of the vehicle, and wherein the physiological sensor is configured to output at least one of a movement output, an accelerometer output, a gyroscopic output, a pressure output, a body position output, a LIDAR output, a location reading, a blood alcohol reading, or a movement of a chest or abdomen of the individual.

A twentieth aspect can include the system of any of the eleventh aspect to the nineteenth aspect, wherein the one or more sensors comprise a vital data sensor, and wherein the vital data sensor is configured to output at least one of a respiration rate, an electroencephalogram output, a temperature reading, a blood pressure reading, a heart rate, a skin conductance reading, a blood oxygen level, or a blood sugar level.

A twenty first aspect can include the system of any of the eleventh aspect to the twentieth aspect, further comprising: a communication system in signal communication with the analysis unit, wherein the communication system is configured to send an indication of the mental state or the physical state of the individual to a remote device.

In a twenty second aspect, a blood alcohol concentration monitoring system associated with a vehicle, the system comprises: a physiological sensor disposed within a steering wheel of the vehicle, wherein the physiological sensor is configured to measure one or more physiological parameters of a driver when the driver holds the steering wheel; and an analysis unit, wherein the analysis unit is configured to receive the one or more physiological parameters and determine a blood alcohol concentration of the driver.

For purposes of the disclosure herein, the term “comprising” includes “consisting” or “consisting essentially of” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, e.g., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. As used herein, the term “and/or” can mean one, some, or all elements depicted in a list. As an example, “A and/or B” can mean A, B, or a combination of A and B. Use of broader terms such as comprises, includes, having, etc. should be understood to provide support for narrower terms such as consisting of, consisting essentially of, comprised substantially of, etc.

What is claimed is:

1. A system for determining a blood alcohol concentration of an individual, the system comprising:
one or more sensors, wherein the one or more sensors are configured to measure a physiological parameter of the individual; and
an analysis unit configured to receive one or more outputs of the one or more sensors, wherein the analysis unit is configured to determine a mental state or a physical state of the individual based on the one or more outputs of the one or more sensors.

2. The system of claim 1, further comprising a vehicle, wherein the one or more sensors and the analysis unit are part of the vehicle.

3. The system of claim 1, wherein the one or more sensors comprise an image capture sensor, a vital data sensor, a medical sensor, or a psychological monitoring sensor.

4. The system of claim 1, wherein the one or more sensors comprise an image capture sensor, and wherein the image capture sensor is configured to output at least one of an image or a video.

5. The system of claim 1, wherein the one or more sensors comprise a physiological sensor, and wherein the physiological sensor is configured to output at least one of a movement output, an accelerometer output, a gyroscopic output, a pressure output, a body position output, a LIDAR output, a location reading, a blood alcohol reading, or a movement of a chest or abdomen of the individual.

6. The system of claim 1, wherein the one or more sensors comprise a vital data sensor, and wherein the vital data sensor is configured to output at least one of a respiration rate, an electroencephalogram output, a temperature reading, a blood pressure reading, a heart rate, a skin conductance reading, a blood oxygen level, or a blood sugar level.

7. The system of claim 1, further comprising:
a communication system in signal communication with the analysis unit, wherein the communication system is configured to send an indication of the mental state or the physical state of the individual to a remote device.

8. The system of claim 1, wherein the analysis unit comprises a neural network configured to accept the one or more outputs of the one or more sensors, and predict the physical state of the individual.

9. The system of claim 8, wherein the physical state comprises a blood alcohol content of the individual.

10. The system of claim 9, wherein the analysis unit is further configured to lock an ignition of a vehicle when the blood alcohol content of the individual is above a threshold.

11. A method of monitoring a state of a driver, the method comprising:
monitoring at least one of a physiological parameter, a facial feature, or a psychological parameter of the driver using one or more sensors disposed within a vehicle;
receiving, by an analysis unit, at least one output of the one or more sensors; and
determining, by the analysis unit, a mental state or a physical state of the driver based on the at least one output.

12. The method of claim 11, wherein determining the mental state or the physical state comprises:
inputting the at least one output into a machine learning model;
determining a blood alcohol concentration (BAC) level of the driver as an output of the machine learning model; and
outputting the BAC level of the driver.

13. The method of claim 12, further comprising:
determining that the BAC level of the driver is above a threshold;
monitoring a behavior of the driver;
determining that the behavior of the driver indicates that the driver is unstable; and
sending an alert to a response management unit in response to determining that the behavior of the driver is unstable.

14. The method of claim 12, wherein the one or more sensors comprise an image capture system, and wherein determining the BAC level of the driver comprises:
capturing one or more images of the driver;
detecting one or more facial features of the driver within the images;
determine an indication of the one or more facial features; and
determining the BAC level of the driver using the one or more facial features in an analysis model.

15. The method of claim 14, further comprising:
using one or more physiological sensor outputs or one or more vital sensor outputs with the one or more facial features in the analysis model.

16. The method of claim 12, further comprising:
determining that the BAC level of the driver is above a threshold;
presenting one or more questions to the driver on a display interface;
receive one or more answers from the driver in response to the one or more questions;
inputting the one or more answers to a psychological analysis model; and
determining a psychological state of the driver based on an output of the psychological analysis model.

17. The method of claim 11, wherein the one or more sensors comprise an image capture sensor configured to output at least one of an image or a video, a vital data sensor, a medical sensor, or a psychological monitoring sensor.

18. The method of claim 11, wherein the one or more sensors comprise a physiological sensor disposed within a steering wheel of the vehicle, and wherein the physiological sensor is configured to output at least one of a movement output, an accelerometer output, a gyroscopic output, a pressure output, a body position output, a LIDAR output, a location reading, a blood alcohol reading, or a movement of a chest or abdomen of the individual, and wherein the one or more sensors comprise a vital data sensor, and wherein the vital data sensor is configured to output at least one of a respiration rate, an electroencephalogram output, a temperature reading, a blood pressure reading, a heart rate, a skin conductance reading, a blood oxygen level, or a blood sugar level.

19. The method of claim 11, further comprising:
a communication system in signal communication with the analysis unit, wherein the communication system is configured to send an indication of the mental state or the physical state of the individual to a remote device.

20. A blood alcohol concentration monitoring system associated with a vehicle, the system comprising:
a physiological sensor disposed within a steering wheel of the vehicle, wherein the physiological sensor is configured to measure one or more physiological parameters of a driver when the driver holds the steering wheel; and
an analysis unit, wherein the analysis unit is configured to receive the one or more physiological parameters and determine a blood alcohol concentration of the driver.
